(19) United States
(12) Patent Application Publication
(10) Pub. No.: US 2025/0135192 A1
(43) Pub. Date: May 1, 2025

(54) INTEGRATED ELECTRODE FOR MEDICAL DEVICE

(71) Applicant: Greatbatch Ltd., Clarence, NY (US)

(72) Inventors: Hugh D. Hestad, Edina, MN (US); Christopher Mauhar, Maple Grove, MN (US)

(21) Appl. No.: 18/921,125

(22) Filed: Oct. 21, 2024

Related U.S. Application Data
(60) Provisional application No. 63/545,959, filed on Oct. 27, 2023.

(51) Int. Cl. A61N 1/05

(52) U.S. Cl. CPC A61N 1/05

(57) ABSTRACT

In various examples, an electrode for a medical device is described. The medical device includes a lead. The electrode includes a tubular ring formed from a conductive material. The ring extending around a perimeter of the electrode. The ring defines a void within the ring. The void is configured to accept overmolded material of the lead therein to anchor the electrode within the lead. An electrode surface is associated with the ring. The electrode surface is configured to contact and stimulate tissue of a patient with the lead implanted within the patient.

### CROSS REFERENCE TO RELATED APPLICATIONS

This application claims the benefit of priority to U.S. Provisional Application Ser. No. 63/545,959, filed on Oct. 27, 2023, entitled “INTEGRATED ELECTRODE,” which is incorporated by reference herein in its entirety.

### BACKGROUND

The present inventive subject matter is directed to a medical device, and more particularly, to an electrode for a lead of a medical device.

Electrical leads and silicone paddle leads often include electrodes disposed within bodies of the leads. When implanted within a patient, these leads often undergo various stresses and manipulation from bending, pulling, twisting, and the like at the implant location within the patient. Sometimes, such stresses and manipulation of the lead within the patient can cause one or more electrodes within the lead to come loose, dislodge, or otherwise at least partially disengage from within the lead. Such disengagement of one or more electrodes can cause the one or more electrodes to short out or otherwise stop working and/or to stimulate a different location or in a different direction within the patient than that which was intended. Another major concern is the potential for an edge of the dislodged electrode to damage soft tissue during routine movements. The damage created by the motion against the free metal edge can result in discomfort, additional pain, or other lasting damage to the patient's anatomy.

Conventional electrodes sometimes include flanges or flaps that help to secure the electrodes within layers and/or materials of a lead. However, such electrodes can still become dislodged when the lead undergoes stresses and/or manipulation within the patient.

### Overview

This overview is intended to provide an overview of subject matter of the present patent document. It is not intended to provide an exclusive or exhaustive explanation of the invention. The detailed description is included to provide further information about the present patent document.

The present inventors have recognized, among other things, that the present subject matter can be used to provide an electrode embedded within a lead in a way to provide mechanical stability within the lead, in particular when the lead undergoes stresses and/or manipulation, to guard against the electrode coming loose from, popping out of, dislodging from, or otherwise disengaging from within the lead. In various examples, the present inventive subject matter is advantageous in that it provides adequate stability of the electrodes with respect to the lead during bending or manipulation of the lead, in some examples, through mechanical integration of the electrode with the carrier material of the lead. In some examples, the present inventive subject matter guards against electrodes coming loose or “popping out” of the lead or paddle during bending or aggressive manipulation of the lead. In various examples, the present inventive subject matter is advantageous in that it provides adequate mechanical attachment to a lead with low manufacturing difficulty. In some examples, the electrode of the present inventive subject matter is advantageous in that it relies on an overmold to integrate the electrode to the paddle lead or lead. To better illustrate the devices described herein, a non-limiting list of examples is provided here:

Example 1 can include subject matter that can include an electrode for a medical device. The medical device includes a lead. The electrode includes a tubular ring formed from a conductive material. The ring extends around a perimeter of the electrode. The ring defines a void within the ring. The void is configured to accept overmolded material of the lead therein to anchor the electrode within the lead. An electrode surface is associated with the ring. The electrode surface is configured to contact and stimulate tissue of a patient with the lead implanted within the patient.

In Example 2, the subject matter of Example 1 is optionally configured such that the ring is at least partially flattened such that the ring is substantially ellipse shaped.

In Example 3, the subject matter of Example 1 or 2 is optionally configured such that the electrode surface is integrally formed with the ring.

In Example 4, the subject matter of any one of Examples 1-3 is optionally configured such that the electrode surface is a substantially flattened portion of the ring.

In Example 5, the subject matter of any one of Examples 1-4 is optionally configured such that the electrode surface is attached to the ring.

In Example 6, the subject matter of Example 5 is optionally configured such that the electrode surface is welded to the ring.

In Example 7, the subject matter of Example 5 or 6 is optionally configured such that the electrode surface is formed into a substantially rectangular shape.

In Example 8, the subject matter of any one of Examples 1-7 is optionally configured such that the ring includes a longitudinal ring axis extending along a length of the ring. The ring is configured to be disposed within the lead such that the longitudinal ring axis extends substantially parallel to a longitudinal lead axis.

In Example 9, the subject matter of any one of Examples 1-8 is optionally configured such that the ring includes a longitudinal ring axis extending along a length of the ring. The ring is configured to be disposed within the lead such that the longitudinal ring axis extends substantially perpendicular to a longitudinal lead axis.

In Example 10, the subject matter of any one of Examples 1-9 optionally includes a crimp tube attached to the ring, the crimp tube configured to receive and electrically couple to a conductor of the lead.

Example 11 can include, or can optionally be combined with any one of Examples 1-10 to include subject matter that can include a lead for a medical device. The lead includes a lead body. An electrode is disposed within the lead body. The electrode includes a tubular ring formed from a conductive material. The ring extends around a perimeter of the electrode. The ring defines a void within the ring. The void is configured to accept overmolded material of the lead therein to anchor the electrode within the lead. An electrode surface is associated with the ring. The electrode surface is configured to contact and stimulate tissue of a patient with the lead implanted within the patient.

In Example 12, the subject matter of Example 11 is optionally configured such that the ring of the electrode is at least partially flattened such that the ring is substantially ellipse shaped.

In Example 13, the subject matter of Example 11 or 12 is optionally configured such that the electrode surface is integrally formed with the ring.

In Example 14, the subject matter of Example 13 is optionally configured such that the electrode surface is a substantially flattened portion of the ring.

In Example 15, the subject matter of any one of Examples 11-14 is optionally configured such that the electrode surface is attached to the ring.

In Example 16, the subject matter of Example 15 is optionally configured such that the electrode surface is welded to the ring.

In Example 17, the subject matter of Example 15 or 16 is optionally configured such that the electrode surface is formed into a substantially rectangular shape.

In Example 18, the subject matter of any one of Examples 11-17 is optionally configured such that the ring includes a longitudinal ring axis extending along a length of the ring. The ring is configured to be disposed within the lead such that the longitudinal ring axis extends substantially parallel to a longitudinal lead axis.

In Example 19, the subject matter of any one of Examples 11-18 is optionally configured such that the ring includes a longitudinal ring axis extending along a length of the ring. The ring is configured to be disposed within the lead such that the longitudinal ring axis extends substantially perpendicular to a longitudinal lead axis.

In Example 20, the subject matter of any one of Examples 11-19 optionally includes a conductor extending within the lead. The electrode includes a crimp tube attached to the ring. The crimp tube is configured to receive and electrically couple to a conductor of the lead.

### BRIEF DESCRIPTION OF THE DRAWINGS

FIG. 1 is a perspective view of a paddle lead including electrodes in accordance with at least one example of the invention.

FIG. 2 is a perspective view of an electrode in accordance with at least one example of the invention.

FIG. 3 is a perspective view of an electrode in accordance with at least one example of the invention.

FIG. 4 is a perspective view of an electrode in accordance with at least one example of the invention.

FIG. 5 is a a perspective view of an electrode in accordance with at least one example of the invention.

FIG. 6 is a perspective view of a paddle lead including electrodes in accordance with at least one example of the invention.

FIG. 7 is a perspective view of a lead including electrodes in accordance with at least one example of the invention.

### DETAILED DESCRIPTION

The present invention relates generally to providing an electrode for a lead within a patient. More specifically, the present invention relates to an electrode embedded within a lead in a way to provide mechanical stability within the lead, in particular when the lead undergoes stresses and/or manipulation, to guard against the electrode coming loose from, popping out of, dislodging from, or otherwise disengaging from within the lead.

The present inventive subject matter, in some examples, includes an electrical lead with electrodes that are embedded in the lead. In various examples, various types of leads can utilize the electrodes of the present inventive subject matter, including, but not limited to a paddle lead, a cuff lead, a cylindrical body lead, and a flat pad lead, to name a few. Although the examples described herein refer mostly to a paddle lead and a cylindrical body (percutaneous) lead, it should be understood that the concepts described herein can refer to lead types other than paddle leads and cylindrical body leads and that the use of the example electrodes of the present inventive subject matter in such other lead types is contemplated herein. In some examples, the electrodes are configured to provide adequate stability of the electrodes with respect to the lead during bending or manipulation of the lead. In some examples, mechanical integration of the electrode with the carrier material of the lead provides that stability. In this way, in some examples, the electrodes will not come loose or “pop out” of the lead or paddle during bending or aggressive manipulation of the lead.

In some examples, the electrodes of the present inventive subject matter can provide adequate mechanical attachment to a lead with low manufacturing difficulty. In some examples, the electrode of the present inventive subject matter relies on an overmold to integrate the electrode to the paddle lead or lead. The electrode of the present inventive subject matter, in some examples, is integrated with the lead paddle or lead body by overmolding in a way that adequately secures the electrode to the lead paddle or lead body. In some examples, as an alternative to overmolding, a liquid silicone rubber material can be used to fill in around the electrode and then a back sheet can be applied to secured the electrode to the lead paddle or lead body.

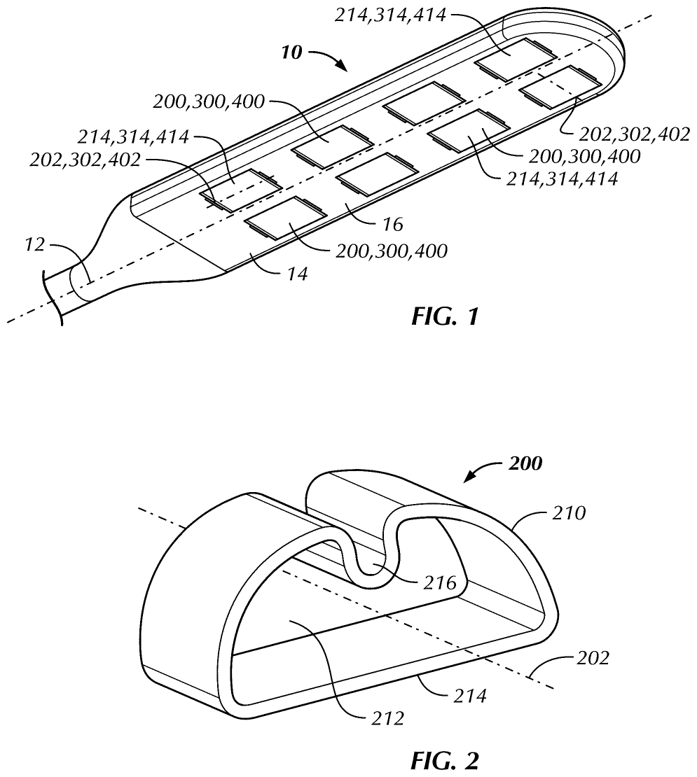

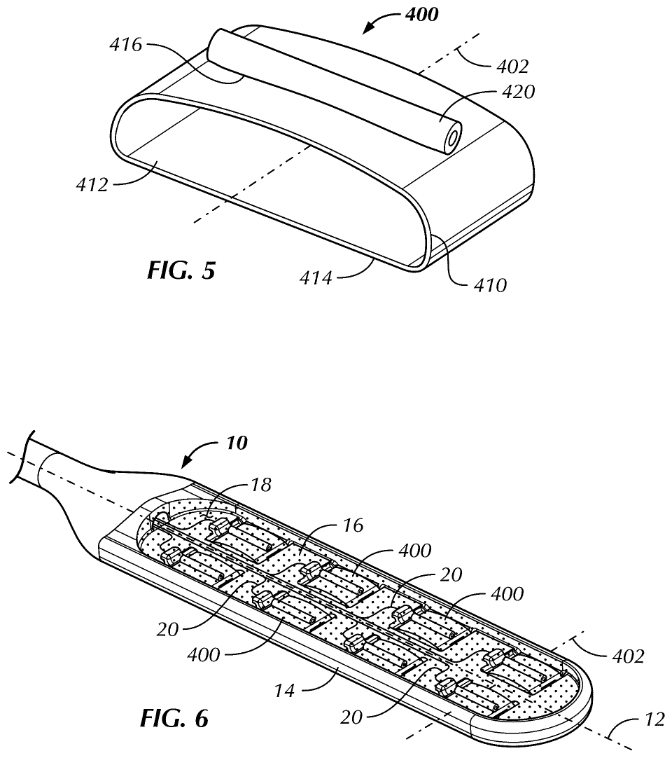

Referring now to the drawings wherein like reference numerals identify similar structural elements or features of the subject invention, there is illustrated in FIGS. 1 and 6 a paddle lead 10 that, in some examples, includes a longitudinal lead axis 12 and a paddle 14 at a distal end of the paddle lead 10. In some examples, as shown specifically in FIG. 1, the paddle lead 10 includes one or more electrodes 200, 300, 400 within the paddle 14, the one or more electrodes 200, 300, 400 each including an electrode surface 214, 314, 414 exposed from a surface of the paddle 14 to allow for the one or more electrodes 200, 300, 400 to stimulate tissue of a patient contacted by the electrode surface 214, 314, 414 with the paddle lead 10 implanted within the patient. In some examples, the one or more electrodes 200, 300, 400 are placed in a carrier or face sheet 16 to define the positions, the spacing, and location of the one or more electrodes 200, 300, 400. An electrical conductor, wire, or cable 20 (see FIG. 6) is attached to each of the one or more electrodes 200, 300, 400, in some examples, via direct welding or the addition of a crimp tube to reduce a heat affected zone on the conductor, wire, or cable 20. Once the one or more electrodes 200, 300, 400 are positioned within the face sheet 16 and the one or more conductors 20 routed and attached to the corresponding one or more electrodes 200, 300, 400, overmold material 18 (see FIG. 6) is inserted into the paddle 14 of the lead 10 to flow and cure in and around the one or more electrodes 200, 300, 400, thereby encapsulating and anchoring the one or more electrodes 200, 300, 400 within the paddle 14 of the lead 10. In some examples, the one or more electrodes 200, 300, 400 are placed with the conductors 20 and then overmolded or potted to introduce the overmold material 18 inside the electrode area of the paddle 14 to provide the mechanical integration. The overmold material 18, in some examples, is also adhered or integrated to the carrier or face sheet 16.

In the example shown, the paddle 14 includes eight electrodes 200, 300, 400 arranged in two columns of four electrodes 200, 300, 400. However, this is not intended to be limiting. In other examples, other numbers and configurations of electrodes 200, 300, 400 within the paddle 14 are contemplated. For instance, the paddle 14, in some examples, can include three or more columns of electrodes 200, 300, 400. In other examples, the electrodes 200, 300, 400 can be arranged in rows in addition to being arranged in columns, such as is shown in FIG. 1 where the electrodes 200, 300, 400 are positioned in four rows of two electrodes 200, 300, 400. In still other examples, the electrodes 200, 300, 400 in adjacent columns can be offset from one another. Various configurations, positioning, and numbers of the electrodes 200, 300, 400 are contemplated herein in various paddle leads 10, depending upon the ultimate application and requirements of the paddle lead 10. Moreover, while FIG. 1 represents the electrodes 200, 300, 400 at each of the electrode locations within the paddle 14 of the paddle lead 10, it should be understood that not all of the electrodes 200, 300, 400 are present in each of the electrode locations at the same time. Instead, it is meant to demonstrate that any one of the below-described electrodes 200, 300, 400 can be disposed in each of the electrode positions of the paddle 14 of the paddle lead 10. In some examples, the paddle lead 10 can include all of the same type of electrode 200, 300, 400. In other examples, the paddle lead 10 can include a combination of different types of electrodes 200, 300, 400.

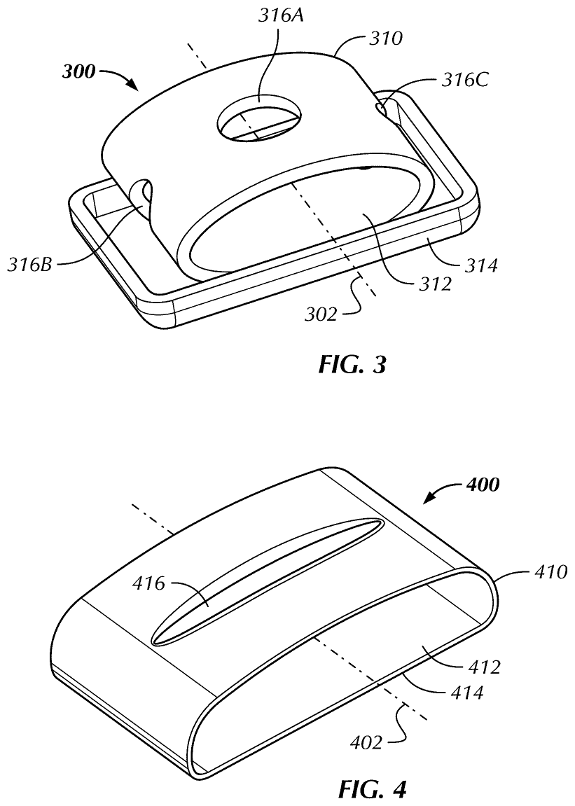

Referring to FIG. 2, the electrode 200, in some examples, is configured to be used in one or more of the electrode positions of the paddle lead 10 of FIG. 1. The electrode 200, in some examples, includes a tubular ring 210 formed from a conductive material. In some examples, the ring 210 extends around a perimeter of the electrode 200 and defines a void 212 within the ring 210. The ring 210, in some examples, includes a longitudinal ring axis 202 extending along a length of the electrode 200. In some examples, the longitudinal ring axis 202 extends through the void 212 of the ring 210. In some examples, the ring 210 includes an electrode surface 214 associated with the ring 210. In some examples, the electrode surface 214 is configured to contact and stimulate tissue of a patient with the lead 10 implanted within the patient. In some examples, a portion of the ring 210 is formed into the electrode surface 214. In further examples, the electrode surface 214 is integrally formed with the ring 210.

In various examples, the ring 210 can be formed into various shapes, depending upon the application for the paddle lead 10; the size, shape, and/or configuration of the paddle 14 within which the electrode 200 is to be disposed; the location of implantation for the paddle lead 10; or the like. In some examples, the electrodes 200 can be made from rings formed into various shapes or extruded and cut to form. In some examples, the electrode 200 has a generally tubular shape with one surface (the electrode surface 214) designated for electrical stimulation.

In some examples, the ring 210 is somewhat flattened or compressed into a non-circular shape. In further examples, the electrode surface 214 is substantially flattened, such that the electrode surface 214 is a substantially flattened portion of the ring 210. In some examples, the electrode 200 is positioned within the face sheet 16 of the paddle lead 10 such that the substantially flattened electrode surface 214 is within the paddle 14 but exposed to an exterior of the lead 10 to allow for the electrode surface 214 to contact and ultimately stimulate the tissue of the patient with the lead 10 implanted within the patient.

In some examples, the electrode 210 is formed into a substantially semicircular shape in cross section, as shown in FIG. 2. In some examples, a channel 216 can be formed within a portion of the ring 210 of the electrode 200 to allow for welding, crimping, or otherwise connecting of a conductor to the electrode 200. That is, in some examples, an end of a conductor can be placed within the channel 216 (with or without a crimp tube attached to the end of the conductor), and then the end of the conductor can be attached to the ring 210 within the channel 216, for instance, by laser welding, brazing, crimping (that is, compressing walls of the channel 216 into contact with the end of the conductor), or a combination thereof. In some examples, the conductor is laser welded to the ring 210. In further examples, the conductor is also mechanically connected to the ring 210 in addition to being laser welded.

In some examples, the void 212 is configured to accept overmold material 18 (see FIG. 6) of the lead 10 therein in order to anchor the electrode 200 within the lead 10. That is, the electrode 200 can be placed within the face sheet 16 of the paddle 14 at the desired location prior to overmolding of the lead 10. Then, once the one or more electrodes 200 are disposed within the paddle 14 of the lead 10, the lead 10 can be overmolded such that the overmold material 18 (such as, for instance, a polymeric material) is molded over the face sheet 16 of the paddle 14 in which the one or more electrodes 200 are positioned, with the overmold material 18 filling in around the one or more electrodes 200 and flowing within the void 212 of each of the one or more electrodes 200 and effectively submerging the one or more electrodes 200 within the overmold material 18 and embedding the one or more electrodes 200 within the paddle 14 of the paddle lead 10. In this way, with the overmold material 18 substantially surrounding each of the one or more electrodes 200 (except for the exposed electrode surface 214 of each of the one or more electrodes 200) and being disposed within the void 212 of each of the one or more electrodes 200, the one or more electrodes 200 are encapsulated within the overmold material 18 of the paddle 14 and anchored within the paddle 14 to inhibit the one or more electrodes 200 from dislodging from within the paddle 14 of the lead 10 when the lead 10 undergoes twisting, bending, or manipulation with movement of the patient within whom the lead 10 is implanted.

In some examples, the ring 210 is configured to be disposed within the lead 10 such that the longitudinal ring axis 202 extends substantially parallel to a longitudinal lead axis 12. In other examples, the ring 210 is configured to be disposed within the lead 10 such that the longitudinal ring axis 202 extends substantially perpendicular to a longitudinal lead axis 12. In still other examples, the ring 210 can be configured to be disposed within the lead 10 such that the longitudinal ring axis 202 extends at any angle between perpendicular and parallel to a longitudinal lead axis 12. Various factors can help determine at what angle (between the longitudinal lead axis 12 and the longitudinal ring axis 202) the electrode 200 should be placed within the paddle 14 including, but not limited to, the application of the lead 10; the likely direction of bending, twisting, or other movement of the lead 10 when implanted and what angle is most likely to provide proper anchoring of the electrode 200 within the lead 10; routing of the conductor 20 to the electrode 200; or the like.

Referring to FIG. 3, the electrode 300, in some examples, is configured to be used in one or more of the electrode positions of the paddle lead 10 of FIG. 1. The electrode 300, in some examples, includes a tubular ring 310 formed from a conductive material. In some examples, the ring 310 extends around a perimeter of the electrode 300 and defines a void 312 within the ring 310. The ring 310, in some examples, includes a longitudinal ring axis 302 extending along a length of the electrode 300. In some examples, the longitudinal ring axis 302 extends through the void 312 of the ring 310. In some examples, the ring 310 includes an electrode surface 314 associated with the ring 310. In some examples, the electrode surface 314 is configured to contact and stimulate tissue of a patient with the lead 10 implanted within the patient. In some examples, the electrode surface 314 includes a separate component that is attached to the ring 310. In further examples, the electrode surface 314 is attached to the ring 310 by welding the electrode surface 314 to the ring 310. In other examples, the electrode surface 314 is attached to the ring 310 by stamping the electrode surface 314 to the ring 310. The electrode surface 314, in various examples, can be formed into various shapes, depending upon the intended application for the lead 10. In some examples, the electrode surface 314 is formed into a substantially rectangular shape, as shown in FIG. 3. However, this is not intended to be limiting as the electrode surface can take shapes other than rectangular, such as, but not limited, to circular, elliptical, triangular, rhombus-shaped, diamond-shaped, pentagonal, hexagonal, octagonal, or the like. In this way, forming the electrode surface 314 separately from the ring 310 and then attaching the electrode surface 314 to the ring 310 allows for greater flexibility for determining the shape of the electrode surface 314 than an electrode with an electrode surface that is integrally formed with an electrode ring, such as the above-described example electrode 200 of FIG. 2.

In various examples, the ring 310 can be formed into various shapes, depending upon the application for the paddle lead 10; the size, shape, and/or configuration of the paddle 14 within which the electrode 300 is to be disposed; the location of implantation for the paddle lead 10; or the like. In some examples, the electrodes 300 can be made from rings formed into various shapes or extruded and cut to form. In some examples, the electrode 300 has a generally tubular shape. In some examples, the ring 310 is somewhat flattened or compressed into a non-circular shape. In further examples, the electrode surface 314 is substantially flat and attached to a portion of the ring 310. In some examples, the electrode 300 is positioned within the face sheet 16 of the paddle lead 10 such that the substantially flattened electrode surface 314 is within the paddle 14 but exposed to an exterior of the lead 10 to allow for the electrode surface 314 to contact and ultimately stimulate the tissue of the patient with the lead 10 implanted within the patient.

In some examples, the electrode 310 is formed into a substantially elliptical shape in cross section, as shown in FIG. 3. In some examples, the ring 310 includes a top access hole 316A that can allow access within the void 312 from a top of the ring 310 for routing of a conductor therethrough and attachment of the conductor to an interior surface of the ring 310, tool access for welding or stamping together of the ring 310 and the electrode surface 314, and/or additional ingress and encapsulation of the ring 310 for the overmold material 18 in order to provide additional anchoring and mechanical fixation of the electrode 300 within the paddle 14. In some examples, the ring 310 includes at least one side access hole 316B, 316C that can allow access within the void 312 from a side of the ring 310 for routing of the conductor therethrough and attachment of the conductor to an interior surface of the ring 310, tool access for welding or stamping together of the ring 310 and the electrode surface 314, and/or additional ingress and encapsulation of the ring 310 for the overmold material 18 in order to provide additional anchoring and mechanical fixation of the electrode 300 within the paddle 14. In further examples, the ring 310 includes both of the side access holes 316B, 316C, to allow for insertion of the conductor through the ring 310 or insertion of a crimp tube (similar to crimp tube 420 described below) through the side access holes 316B, 316C and attachment (for instance, using laser welding, brazing, or the like) of the crimp tube to the ring 310 at the one or both of the locations of the side access holes 316B, 316C. In this way, in some examples, the end of the conductor can be inserted within the crimp tube and attached thereto (through welding, brazing, crimping, or the like), and, with attachment of the crimp tube to the ring 310, an electrical connection is made from the conductor to the crimp tube to the ring 310 and finally to the electrode surface 314.

In some examples, the spaced-apart access holes 316B, 316C are centered along vertices of opposed semi-major axes of the elliptically-shaped ring 310, which puts them at a significant distance from each other. That way, a crimp tube, for example the crimp tube (such as the crimp tube 420 shown in FIG. 5), connected to the end of an electrical conductor is moved into and through one of the holes 316B, 316C and then contacts the ring 310 at the other of the holes 316B, 316C to span the distance between the holes. The crimp tube is then secured to the ring electrode 310 to provide a highly-stable connection for the electrical conductor inside a lumen of the crimp tube in turn connected to the ring 310 at the holes 316B, 316C. In some examples, the ring 310 has a flattened surface where it contacts the inner surface of the electrode surface 314 to increase the surface area of the connection of the ring 310 to the electrode 314. The connection of the crimp tube to the ring 310 at the holes 316B, 316C is spaced laterally outwardly a significant distance from where the ring 310 is connected to the electrode 314 to provide the highly-stable connection between the crimp tube and/or electrical conductor assembly and the ring 310.

In some examples, the void 312 is configured to accept overmold material 18 (see FIG. 6) of the lead 10 therein in order to anchor the electrode 300 within the lead 10. That is, the electrode 300 can be placed within the face sheet 16 of the paddle 14 at the desired location prior to overmolding of the lead 10. Then, once the one or more electrodes 300 are disposed within the paddle 14 of the lead 10, the lead 10 can be overmolded such that the overmold material 18 (such as, for instance, a polymeric material) is molded over the face sheet 16 of the paddle 14 in which the one or more electrodes 300 are positioned, with the overmold material 18 filling in around the one or more electrodes 300 and flowing within the void 312 of each of the one or more electrodes 300 and effectively submerging the one or more electrodes 300 within the overmold material 18 and embedding the one or more electrodes 300 within the paddle 14 of the paddle lead 10. In this way, with the overmold material 18 substantially surrounding each of the one or more electrodes 300 (except for the exposed electrode surface 314 of each of the one or more electrodes 300) and being disposed within the void 312 of each of the one or more electrodes 300, the one or more electrodes 300 are encapsulated within the overmold material 18 of the paddle 14 and anchored within the paddle 14 to inhibit the one or more electrodes 300 from dislodging from within the paddle 14 of the lead 10 when the lead 10 undergoes twisting, bending, or manipulation with movement of the patient within whom the lead 10 is implanted.

In some examples, the ring 310 is configured to be disposed within the lead 10 such that the longitudinal ring axis 302 extends substantially parallel to a longitudinal lead axis 12. In other examples, the ring 310 is configured to be disposed within the lead 10 such that the longitudinal ring axis 302 extends substantially perpendicular to a longitudinal lead axis 12. In still other examples, the ring 310 can be configured to be disposed within the lead 10 such that the longitudinal ring axis 302 extends at any angle between perpendicular and parallel to a longitudinal lead axis 12. Various factors can help determine at what angle (between the longitudinal lead axis 12 and the longitudinal ring axis 202) the electrode 300 should be placed within the paddle 14 including, but not limited to, the application of the lead 10; the likely direction of bending, twisting, or other movement of the lead 10 when implanted and what angle is most likely to provide proper anchoring of the electrode 300 within the lead 10; routing of the conductor 20 to the electrode 300; or the like.

Referring to FIGS. 4-6, the electrode 400, in some examples, is configured to be used in one or more of the electrode positions of the paddle lead 10 of FIGS. 1 and 6. The electrode 400, in some examples, includes a tubular ring 410 formed from a conductive material. In some examples, the ring 410 extends around a perimeter of the electrode 400 and defines a void 412 within the ring 410. The ring 410, in some examples, includes a longitudinal ring axis 402 extending along a length of the electrode 400. In some examples, the longitudinal ring axis 402 extends through the void 412, generally centered in the ring 410. In some examples, the ring 410 includes an electrode surface 414 associated with the ring 410. In some examples, the electrode surface 414 is configured to contact and stimulate tissue of a patient with the lead 10 implanted within the patient. In some examples, a portion of the ring 410 is formed into the electrode surface 414. In further examples, the electrode surface 414 is integrally formed with the ring 410.

In various examples, the ring 410 can be formed into various shapes, depending upon the application for the paddle lead 10; the size, shape, and/or configuration of the paddle 14 within which the electrode 400 is to be disposed; the location of implantation for the paddle lead 10; or the like. In various examples, the electrodes 400 can be made from tubular rings formed into various shapes or extruded and cut to form. In some examples, the electrode 400 has a generally tubular shape with one surface (the electrode surface 414) designated for electrical stimulation. In some examples, the ring 410 is somewhat flattened or compressed into a non-circular shape. In further examples, the electrode surface 414 is substantially flattened, such that the electrode surface 414 is a substantially flattened portion of the ring 410. In some examples, the electrode 400 is positioned within the face sheet 16 of the paddle lead 10 such that the substantially flattened electrode surface 414 is within the paddle 14 but exposed to an exterior of the lead 10 to allow for the electrode surface 414 to contact and ultimately stimulate the tissue of the patient with the lead 10 implanted within the patient.

In some examples, the tubular ring 410 is formed into a substantially elliptical shape in cross section, as shown in FIGS. 4 and 5. In some examples, a channel 416 can be formed within a portion of the ring 410 of the electrode 400 to allow for welding, crimping, or otherwise connecting of a conductor 20 to the electrode 400. In some examples, the channel 416 includes a dent, indentation, groove, or other hollowed out feature formed in a top surface of the ring 410. In some examples, an end of a conductor 20 can be placed within the channel 416, and then the end of the conductor 20 can be attached directly to the ring 410 within the channel 416, for instance, by laser welding, brazing, or the like. In some examples, a shape of the channel 416 allows for increased surface contact and/or a more stable mechanical and electrical connection between the ring 410 and the end of the conductor 20 than would otherwise be possible without the channel 416 and just attaching the end of the conductor 20 to the ring 410 without the channel 416.

In some examples, the channel 416 includes an elliptical shape extending into an upper surface of the ring 410, spaced above the flattened electrode surface 414. Vertices at the opposed semi-major axes of the elliptically-shaped channel 416 are aligned along a channel axis that is spaced above and substantially perpendicular to the ring axis 402. In an example, the end of an electrical conductor (not shown) is laid in the channel 416 so that the longitudinal axis of the conductor is aligned with or substantially parallel to the longitudinal axis of the channel 416. Then, the electrical conductor is connected to the tubular ring 410 along at least one, and preferably both sides of the channel 416, to provide a highly-stable connection for the electrical conductor nested in the channel 416 of the ring 410. A suitable connection is made by a laser weld or a solder connection.

In other examples, the crimp tube 420 is connected to the end of an electrical conductor, and the crimp tube 420 and/or conductor assembly is laid in the channel 416 so that the longitudinal axis of the crimp tube 420 is aligned with or substantially parallel to the longitudinal axis of the channel 416. Then, in some examples, the crimp tube 420 is connected to the tubular ring 410 along at least one, and preferably both sides of the channel 416, to provide a highly-stable connection for the electrical conductor inside the lumen of the crimp tube 420 in turn nested in the channel 416 of the ring 410. A suitable connection is made by a laser weld or a solder connection. That said, in other examples, such a configuration in which the conductor 20 is attached directly to a ring without the channel is also contemplated herein.

In some examples, instead of attaching the end of the conductor 20 directly to the ring 410, the crimp tube 420 can be used, as shown in FIG. 5. In some examples, the crimp tube 420 is attached to the ring 410, with the crimp tube 420 being configured to receive and electrically couple to the conductor 20 of the lead 10. The crimp tube 420, in some examples, is attached to the end of the conductor 20 (by crimping, welding, brazing, etc., or a combination thereof) and then the crimp tube 420 is attached to an exterior of the ring 410. In some examples, the crimp tube 420 is placed within the channel 416 for attachment (for instance, using laser welding, brazing, or the like) of the crimp tube 420 to the ring 410 along at least a portion of the channel 416. In some examples, the shape of the channel 416 allows for increased surface contact and/or a more stable mechanical and electrical connection between the ring 410 and the crimp tube 420 than would otherwise be possible without the channel 416 and just attaching the crimp tube 420 to the ring 410 without the channel 416. In some examples, the channel 416 assists in locating the crimp tube 420 with respect to the ring 410 during attachment of the crimp tube 420 to the ring 410. That said, in other examples, such a configuration in which the crimp tube 420 is attached directly to a ring without the channel is also contemplated herein. In this way, in some examples, the end of the conductor 20 can be inserted within the crimp tube 420 and attached thereto (through welding, brazing, crimping, or the like), and, with attachment of the crimp tube 420 to the ring 410, an electrical connection is made from the conductor to the crimp tube 420 to the ring 410 and finally to the electrode surface 414.

In some examples, the void 412 is configured to accept overmold material 18 of the lead 10 therein in order to anchor the electrode 400 within the lead 10. That is, the electrode 400 can be placed within the face sheet 16 of the paddle 14 at the desired location prior to overmolding of the lead 10. Then, once the one or more electrodes 400 are disposed within the paddle 14 of the lead 10, the lead 10 can be overmolded such that the overmold material 18 (such as, for instance, a polymeric material) is molded over the face sheet 16 of the paddle 14 in which the one or more electrodes 400 are positioned, with the overmold material 18 filling in around the one or more electrodes 400 and flowing within the void 412 of each of the one or more electrodes 400 and effectively submerging the one or more electrodes 400 within the overmold material 18 and embedding the one or more electrodes 400 within the paddle 14 of the paddle lead 10. In this way, with the overmold material 18 substantially surrounding each of the one or more electrodes 400 (except for the exposed electrode surface 414 of each of the one or more electrodes 400) and being disposed within the void 412 of each of the one or more electrodes 400, the one or more electrodes 400 are encapsulated within the overmold material 18 of the paddle 14 and anchored within the paddle 14 to inhibit the one or more electrodes 400 from dislodging from within the paddle 14 of the lead 10 when the lead 10 undergoes twisting, bending, or manipulation with movement of the patient within whom the lead 10 is implanted.

In some examples, such as the example shown in FIG. 6, the ring 410 is configured to be disposed within the lead 10 such that the longitudinal ring axis 402 extends substantially perpendicular to a longitudinal lead axis 12. In other examples, however, the ring 410 can be configured to be disposed within the lead 10 such that the longitudinal ring axis 402 extends substantially parallel to a longitudinal lead axis 12. In still other examples, the ring 410 can be configured to be disposed within the lead 10 such that the longitudinal ring axis 402 extends at any angle between perpendicular and parallel to a longitudinal lead axis 12. Various factors can help determine at what angle (between the longitudinal lead axis 12 and the longitudinal ring axis 402) the electrode 400 should be placed within the paddle 14 including, but not limited to, the application of the lead 10; the likely direction of bending, twisting, or other movement of the lead 10 when implanted and what angle is most likely to provide proper anchoring of the electrode 400 within the lead 10; routing of the conductor 20 to the electrode 400; or the like.

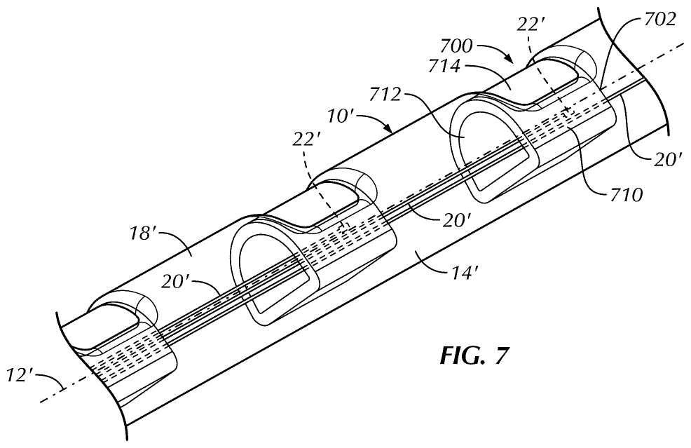

Referring now to FIG. 7, in some examples, a lead 10′ (such as, but not limited to a percutaneous lead) includes a lead body 14′ and a longitudinal lead axis 12′ along the lead body 14′. In some examples, the lead 10′ includes one or more electrodes 700 within the lead body 14′, the one or more electrodes 700 each including an electrode surface 714 exposed from a surface of the lead body 14′ to allow for the one or more electrodes 700 to stimulate tissue of a patient contacted by the electrode surface 714 with the lead 10′ implanted within the patient. In some examples, the one or more electrodes 700 are positioned in a line within the lead body 14′, spaced from one another by a desired distance based on the application and/or implantation location of the lead 10′. An electrical conductor, wire, or cable 20′ is attached to each of the one or more electrodes 700, in some examples, via direct welding or the addition of a crimp tube to reduce a heat affected zone on the conductor, wire, or cable 20′. Once the one or more electrodes 700 are properly positioned and the one or more conductors 20′ routed and attached to the corresponding one or more electrodes 700, overmold material 18′ is molded around the one or more electrodes 700 of the lead 10′ to at least partially form the lead body 14′ and flow and cure in and around the one or more electrodes 700, thereby encapsulating and anchoring the one or more electrodes 700 within the lead body 14′ of the lead 10′. In some examples, the one or more electrodes 700 are placed with the conductors 20 and then overmolded or potted to introduce the overmold material 18′ in and around the one or more electrodes 700 to provide the mechanical integration of the one or more electrodes 700 within the lead body 14′ of the lead 10′.

In the example shown, only three electrodes 700 are shown disposed within the partial representation of the lead 10′. However, this is not intended to be limiting. In other examples, other numbers and configurations of electrodes 700 (more or fewer than three) within the lead body 14′ are contemplated. For instance, in some examples, the lead body 14′ can include eight electrodes 700 disposed proximate a distal end of the lead 10′. In other examples, more or fewer than eight electrodes 700 can be disposed within the lead body 14′ depending upon the intended application for the lead 10′. In still other examples, the one or more electrodes 700 can be located in a portion of the lead 10′ other than the distal end of the lead 10′ or can be located in two or more different locations along the lead. Various configurations, positioning, and numbers of the electrodes 700 are contemplated herein in various leads 10′, depending upon the ultimate application and requirements of the lead 10′.

The electrode 700, in some examples, is configured to be used in one or more of the electrode positions of the lead 10′. The electrode 700, in some examples, includes a tubular ring 710 formed from a conductive material. In some examples, the ring 710 extends around a perimeter of the electrode 700 and defines a void 712 within the ring 710. The ring 710, in some examples, includes a longitudinal ring axis 702 extending along a length of the electrode 700. In some examples, the longitudinal ring axis 702 extends through the void 712 of the ring 710. In some examples, the ring 710 includes an electrode surface 714 associated with the ring 710. In some examples, the electrode surface 714 is configured to contact and stimulate tissue of a patient with the lead 10′ implanted within the patient. In some examples, a portion of the ring 710 is formed into the electrode surface 714. In further examples, the electrode surface 714 is integrally formed with the ring 710.

In various examples, the ring 710 can be formed into various shapes, depending upon the application for the lead 10′; the size, shape, and/or configuration of the lead body 14′ within which the electrode 700 is to be disposed; the location of implantation for the lead 10′; or the like. In some examples, the electrodes 700 can be made from rings formed into various shapes or extruded and cut to form. In some examples, the electrode 700 has a generally tubular shape with one surface (the electrode surface 714) designated for electrical stimulation. In some examples, the ring 710 is compressed into a non-circular shape. In some examples, the electrode 700 is positioned within the lead body 14′ of the lead 10′ such that the electrode surface 714 is within the lead body 14′ but exposed to an exterior of the lead 10′ to allow for the electrode surface 714 to contact and ultimately stimulate the tissue of the patient with the lead 10′ implanted within the patient. In some examples, the lead 10′ is generally rounded with a formed electrode 700 including a curved surface (the electrode surface 714) that is exposed for stimulation and a rear formed tubular geometry with an inner surface that is overmolded to capture or retain the electrode 700 within the lead 10′ during bending or manipulation of the lead 10′. In the example shown in FIG. 7, the electrodes 700 include electrode surfaces 714 configured to stimulate tissue located on only one side of the lead body 14′ such that the lead 10′ is a directional lead. This is not intended to be limiting, however, such that, in various examples, other leads are contemplated herein which are capable of stimulating 360 degrees around the lead down to just a few degrees of stimulation. In still other examples, the lead can include segmented stimulation such that the lead includes two or more stimulation directions with one or more non-stimulation areas (where the electrode surface is not positioned and/or is not exposed to the exterior of the lead) therebetween.

In some examples, the electrode 710 is formed into a substantially semicircular shape in cross section, as shown in FIG. 7. In some examples, the one or more conductors 20′ are routed within the lead body 14′ and in proximity to the one or more rings 710 so that the end of one of the conductors 20′ can be attached to a corresponding one of the one or more rings 710. In some examples, the end of the conductor 20′ is welded to the ring 710 at an attachment point 22′. In the example shown in FIG. 7, the conductors 22′ are routed through voids 712 of the rings 710 with the end of one of the conductors 20′ terminating within each of the rings 710 at an attachment point 22′ where the end of the conductor 20′ is welded or otherwise attached to an interior of the ring 710. In other examples, however, the conductors 20′ need not be routed through the voids 712 of the rings 710 and, instead, can be routed exteriorly past the rings 710 (but still within the lead body 14′), with the conductors 20′ being attached either to an exterior or an interior of the rings 710. In some examples, the conductors 20′ are welded to the rings 710 prior to overmolding. In this way, an electrical connection is made from each of the conductors 20′ to a corresponding one of the rings 710 and finally to the electrode surface 714 of the ring 710.

In some examples, the void 712 is configured to accept the overmold material 18′ of the lead 10′ therein in order to anchor the electrode 700 within the lead 10′. That is, the electrode 700 can be placed within the lead body 14′ at the desired location prior to overmolding of the lead 10′. Then, once the one or more electrodes 700 are properly positioned, the lead body 14′ can be overmolded such that the overmold material 18′ (such as, for instance, a polymeric material) is molded to at least partially form the lead body 14′ in which the one or more electrodes 700 are positioned, with the overmold material 18′ filling in around the one or more electrodes 700 and flowing within the void 712 of each of the one or more electrodes 700 and effectively submerging the one or more electrodes 700 within the overmold material 18′ and embedding the one or more electrodes 700 within the lead body 14′ of the lead 10′. In some examples, the ring 710 is disposed within the lead 10′ such that the longitudinal ring axis 702 extends substantially parallel to the longitudinal lead axis 12′. In this way, with the overmold material 18′ substantially surrounding each of the one or more electrodes 700 (except for the exposed electrode surface 714 of each of the one or more electrodes 700) and being disposed within the void 712 of each of the one or more electrodes 700, the one or more electrodes 700 are encapsulated within the overmold material 18′ of the lead body 14′ and anchored within the lead body 14′ to inhibit the one or more electrodes 700 from dislodging from within the lead body 14′ of the lead 10′ when the lead 10′ undergoes twisting, bending, or manipulation with movement of the patient within whom the lead 10′ is implanted.

The present inventors have recognized various advantages of the subject matter described herein. The present inventors have recognized, among other things, that the present subject matter can be used to provide an electrode embedded within a lead in a way to provide mechanical stability within the lead, in particular when the lead undergoes stresses and/or manipulation, to guard against the electrode coming loose from, popping out of, dislodging from, or otherwise disengaging from within the lead. In various examples, the present inventive subject matter is advantageous in that it provides adequate stability of the electrodes with respect to the lead during bending or manipulation of the lead, in some examples, through mechanical integration of the electrode with the carrier material of the lead. In some examples, the present inventive subject matter guards against electrodes coming loose or “popping out” of the lead or paddle during bending or aggressive manipulation of the lead. In various examples, the present inventive subject matter is advantageous in that it provides adequate mechanical attachment to a lead with low manufacturing difficulty. In some examples, the electrode of the present inventive subject matter is advantageous in that it relies on an overmold to integrate the electrode to the paddle lead or lead. While various advantages of the example systems are listed herein, this list is not considered to be complete, as further advantages may become apparent from the description and figures presented herein.

Although the subject matter of the present patent application has been described with reference to various examples, workers skilled in the art will recognize that changes can be made in form and detail without departing from the scope of the subject matter recited in the below claims.

The above Detailed Description includes references to the accompanying drawings, which form a part of the Detailed Description. The drawings show, by way of illustration, specific examples in which the present apparatuses and methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” or similar are used to refer to an amount that is nearly, almost, or in the vicinity of being equal to a stated amount.

In the appended claims, the terms “including” and “in which” are used as the plain-English equivalents of the respective terms “comprising” and “wherein.” Also, in the following claims, the terms “including” and “comprising” are open-ended, that is, an apparatus or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

1. An electrode for a medical device, the medical device including a lead, the electrode comprising:
a tubular ring formed from a conductive material, the ring extending around a perimeter of the electrode, the ring defining a void within the ring, the void configured to accept overmolded material of the lead therein to anchor the electrode within the lead; and
an electrode surface associated with the ring, the electrode surface being configured to contact and stimulate tissue of a patient with the lead implanted within the patient.

2. The electrode of claim 1, wherein the ring is at least partially flattened such that the ring is substantially ellipse shaped.

3. The electrode of claim 1, wherein the electrode surface is integrally formed with the ring.

4. The electrode of claim 1, wherein the electrode surface is a substantially flattened portion of the ring.

5. The electrode of claim 1, wherein the electrode surface is attached to the ring.

6. The electrode of claim 5, wherein the electrode surface is welded to the ring.

7. The electrode of claim 5, wherein the electrode surface is formed into a substantially rectangular shape.

8. The electrode of claim 1, wherein the ring includes a longitudinal ring axis extending along a length of the ring, the ring being configured to be disposed within the lead such that the longitudinal ring axis extends substantially parallel to a longitudinal lead axis.

9. The electrode of claim 1, wherein the ring includes a longitudinal ring axis extending along a length of the ring, the ring being configured to be disposed within the lead such that the longitudinal ring axis extends substantially perpendicular to a longitudinal lead axis.

10. The electrode of claim 1, comprising a crimp tube attached to the ring, the crimp tube configured to receive and electrically couple to a conductor of the lead.

11. A lead for a medical device, the lead comprising:
a lead body; and
an electrode disposed within the lead body, the electrode including:
a tubular ring formed from a conductive material, the ring extending around a perimeter of the electrode, the ring defining a void within the ring, the void configured to accept overmolded material of the lead therein to anchor the electrode within the lead; and
an electrode surface associated with the ring, the electrode surface being configured to contact and stimulate tissue of a patient with the lead implanted within the patient.

12. The lead of claim 11, wherein the ring of the electrode is at least partially flattened such that the ring is substantially ellipse shaped.

13. The lead of claim 11, wherein the electrode surface is integrally formed with the ring.

14. The lead of claim 13, wherein the electrode surface is a substantially flattened portion of the ring.

15. The lead of claim 11, wherein the electrode surface is attached to the ring.

16. The lead of claim 15, wherein the electrode surface is welded to the ring.

17. The lead of claim 15, wherein the electrode surface is formed into a substantially rectangular shape.

18. The lead of claim 11, wherein the ring includes a longitudinal ring axis extending along a length of the ring, the ring being configured to be disposed within the lead such that the longitudinal ring axis extends substantially parallel to a longitudinal lead axis.

19. The lead of claim 11, wherein the ring includes a longitudinal ring axis extending along a length of the ring, the ring being configured to be disposed within the lead such that the longitudinal ring axis extends substantially perpendicular to a longitudinal lead axis.

20. The lead of claim 11, comprising a conductor extending within the lead, wherein the electrode includes a crimp tube attached to the ring, the crimp tube configured to receive and electrically couple to a conductor of the lead.
